(19) United States
(12) Patent Application Publication
(10) Pub. No.: US 2025/0262241 A1
(43) Pub. Date: Aug. 21, 2025

(54) METHODS TO IMPROVE STABILITY OF VIRUS TRANSDUCTION OF y8 T CELLS AND APPLICATIONS THEREOF

(71) Applicant: UNICET BIOTECH CO. LLC., Beijing (CN)

(72) Inventor: Weiwei Ma, Beijing (CN)

(21) Appl. No.: 18/285,847

(22) PCT Filed: Apr. 6, 2022
(86) PCT No.: PCT/CN2022/085416

§ 371 Date: Oct. 5, 2023

(30) Foreign Application Priority Data
Apr. 6, 2021 (WO) PCT/CN2021/085619

(51) Int. Cl. A61K 35/17; A61K 40/11; A61K 40/31; A61K 40/42; A61P 35/02; C07K 14/705; C07K 14/725; C07K 16/28; C12N 5/0783; C12N 15/86

(52) U.S. Cl. CPC A61K 35/17; A61K 40/11; A61K 40/31; A61K 40/421; A61P 35/02; C07K 14/7051; C07K 14/70517; C07K 14/70521; C07K 14/70578; C07K 16/2812; C07K 16/2827; C12N 5/0636; C12N 15/86; A61K 2239/13; A61K 2239/17; A61K 2239/21; A61K 2239/48; C07K 2317/53; C07K 2317/622; C07K 2319/03; C12N 2501/727; C12N 2510/00; C12N 2740/15043

(57) ABSTRACT

A method of transducing a γδ T cell with a viral vector includes contacting the γδ T cell with the viral vector and an agent capable of inhibiting the innate anti-virus activity of the γδ T cell. A method of preparing CAR-γδ T cells includes providing γδcells and transducing the γδ T cells with a viral vector including a nucleotide sequence encoding a chimeric antigen receptor in the presence of an agent capable of inhibiting the innate anti-virus activity of the γδ T cells. The methods of transducing γδ T cells can increase transduction rate and/or prevent the decrease of transduction rate during subsequent cell expansion process.

### CROSS REFERENCE TO RELATED APPLICATIONS

This application claims priority from PCT international application PCT/CN2021/085619 filed Apr. 6, 2021, which is incorporated herein by reference in its entirety.

### FIELD OF THE INVENTION

The present disclosure relates to a method for transducing γδ T cells. The present disclosure also relates to a method of preparing CAR-γδ T cells and a preparation comprising the CAR-γδ T cells.

### BACKGROUND

Gamma delta T cells (γδ T cells) are a special type of immune cells which exhibit both adaptive and innate immune response features. γδ T cells co-express TCR types of γ chain and S chain and NKG2D (one of the main function receptors expressed on NK cells), thus allowed γδ T cells mimic both T and NK cell functions. In contrast to the conventional αβ T cells which bearing the TCR of α chain and β chain and recognize antigen-derived peptides presented by the MHC molecules (in humans called human leukocyte antigen [HLA]), γδ T cells can recognize and kill pathogens independent of MHC (MHC unrestricted). And at the same time, γδ T cells release various kinds of cytokines to activate other immune cells, such as NKs, macrophages and CD8+ cytotoxic lymphocytes (1). In particular, blood Vγ9Vδ2 T cells (the major γδ T cells subset in the peripheral blood) are capable of responding to microbes, tumors as well as cluster of differentiation CD4+ and CD8+ T cells (2). γδ T cells also exhibit antigen-presenting ability. It has been shown by many studies that Vγ9Vδ2 T cells possessed broadly tumor killing ability. Hence, as unconventional immune cells, γδ T cells acted as the “bridge” of innate and adaptive immune response.

The MHC dependent antigen recognition mode restricted the application of αβ T cells in allogeneic therapy as the risk of GvHD. The MHC unrestricted γδ T cells are considered to be a great candidate for tumor immunotherapy as they can be used for allogeneic transfer without the concern of GvHD. In the last decade, many researchers have begun to investigate the clinical application of γδ T cells in tumor treatment. The safety and efficiency of autologous or allogenic therapy of γδ T cells has been preliminarily proved (3).

The in vitro culture and expansion methods of αβ T cells and γδ T cells are totally different. For αβ T cells, peripheral blood mononuclear cells (PBMCs) were usually isolated using Ficoll-Paque density gradient centrifugation methods and stimulated with CD3/CD28 Dynabeads. In some experiments, T cells were enriched by CD4/CD8 or CD3 positive selection. However, γδ2 cells constitute <5% of PBMC and stimulation with CD3/CD28 Dynabeads results in barely γδ2 T cell expansion. Instead, γ9δ2 T cells can be activated by bisphosphonates such as Zoledronate (ZOL), phosphoantigen such as isopentenyl pyrophosphate (IPP), (E)-4-hydroxy-3-methyl-but-2-enyl pyrophosphate (HMB-PP) or the synthetic phosphoantigen bromohydrin pyrophosphate (BrHPP) et al. (4).

Compared to classical chimeric antigen receptors T cells (CAR-T), the “CAR” modified γδ T cells (CAR-γδ T cells) seemed to perform better according to some pre-clinical research (5,6). However, challenges remain when transforming CAR-γδ T cells into clinical application. The transduction efficiency of primary γδ T cells with large payload lentiviral vectors is very low. Moreover, transduction stability cannot be ensured as CAR positive rate continuously drops along with γδ T expansion, which is not observed in CAR-αβ T cell manufacture process.

### SUMMARY

In one aspect, the present disclosure provides a method of transducing a γδ T cell with a viral vector, comprising: contacting the γδ T cell with i) the viral vector; and ii) an agent capable of inhibiting the innate anti-virus activity of the γδ T cell.

In some embodiments, the γδ T cell is a δ1, δ2 or δ3 T cell.

In some embodiments, the γδ T cell is a γ9δ2 T cell.

In some embodiments, the viral vector is a retroviral vector.

In some embodiments, the viral vector is a lentiviral vector.

In some embodiments, the viral vector is a VSV-G pseudotyped lentiviral vector.

In some embodiments, the agent acts on the NF-κB signaling pathway.

In some embodiments, the agent is an inhibitor of IKKα, IKKβ, IKKε, IκB kinase, TBK1, PKD1, NF-κB, Akt, PKR, TAK1, IRAK1/4 or proteasome.

In some embodiments, the agent is able to: 1) inhibit the phosphorylation of IκBα; 2) inhibit the function of IκB kinase; 3) inhibit the function of Akt; or 4) inhibit the function of NF-κB, p38 and JNK signaling.

In some embodiments, the agent is selected from the group consisting of BX795, BAY11-7082, Curcumin, Dexamethasone, 2-Aminopurine, (5Z)-7-Oxozeaenol, IRAK1/4 Inhibitor I, and Bortezomib.

In some embodiments, the agent capable of inhibiting the innate anti-virus activity of the γδ T cell is BX795.

In some embodiments, the BX795 is used at a concentration between 0.02 μM-60 μM, more preferably 0.2 μM-6 μM, and most preferably 0.4 μM-2 μM.

In some embodiments, the BX795 is used at a concentration no more than 2 μM.

In some embodiments, the BX795 is used at a concentration between 0.2 μM-0.6 μM.

In some embodiments, BAY11-7082 is used at a concentration between 0.1 μM-2000 μM, more preferably 0.5 μM-200 μM, and most preferably 5 μM-100 μM; or BAY11-7082 is used at a concentration between 0.5 μM-50 μM and more preferably 5 μM-50 μM.

In some embodiments, Curcumin is used at a concentration between 0.1 μM-500 μM, more preferably 1 μM-100 μM, and most preferably 2 μM-20 μM; or Curcumin is used at a concentration between 1 μM-100 μM and more preferably 10 μM-100 μM or 1 μM-10 μM.

In some embodiments, Dexamethasone is used at a concentration between 0.01 μM-500 μM, more preferably 0.1 μM-50 μM, and most preferably 1 μM-10 μM; or Dexamethasone is used at a concentration between 0.064 μM-6.4 μM and more preferably 0.64 μM-6.4 μM.

In some embodiments, 2-Aminopurine is used at a concentration between 0.5 μM-5000 μM, more preferably 5 μM-1000 μM, and most preferably 50 μM-500 μM; or 2-Aminopurine is used at a concentration between 5 μM-500 μM and more preferably 50 μM-500 μM.

In some embodiments, (5Z)-7-Oxozeaenol is used at a concentration between 0.01 μM-600 μM, more preferably 0.6 μM-60 μM, and most preferably 0.6 μM-6 μM; or (5Z)-7-Oxozeaenol is used at a concentration between 0.6 μM-60 μM and more preferably 0.6 μM-6 μM.

In some embodiments, IRAK1/4 Inhibitor I is used at a concentration between 0.01 μM-300 μM, more preferably 0.03 μM-30 μM, and most preferably 0.3 μM-3 μM; or IRAK1/4 Inhibitor I is used at a concentration between 0.03 μM-3 μM and more preferably 0.3 μM-3 μM.

In some embodiments, Bortezomib is used at a concentration between 0.002 μM-40 μM, more preferably 0.01 μM-4 μM, and most preferably 0.01 μM-0.4 μM; or Bortezomib is used at a concentration between 0.04 μM-4 μM, such as 0.04 μM.

In some embodiments, the method further comprises culturing the transduced γδ T cell in a medium without the agent capable of inhibiting the innate anti-virus activity of the γδ T cell.

In some embodiments, the viral vector comprises a nucleotide sequence encoding a chimeric antigen receptor (CAR).

In another aspect, the present disclosure provides a method of preparing CAR-γδ T cells, comprising steps of:
1) providing γδ T cells; and
2) transducing the γδ T cells with a viral vector comprising a nucleotide sequence encoding a chimeric antigen receptor in the present of an agent capable of inhibiting the innate anti-virus activity of the γδ T cells.

In some embodiments, step 1) comprises culturing peripheral blood mononuclear cells (PBMCs) in a medium supplemented with IL-2 and ZOL.

In some embodiments, the method further comprises step 3): culturing the transduced γδ T cells in a medium without the agent capable of inhibiting the innate anti-virus activity of the γδ T cells.

In some embodiments, the γδ T cell is a δ1, δ2 or δ3 T cell.

In some embodiments, the γδ T cell is a γ9δ2 T cell.

In some embodiments, the viral vector is a retroviral vector.

In some embodiments, the viral vector is a lentiviral vector.

In some embodiments, the agent acts on the NF-κB signaling pathway.

In some embodiments, the agent is an inhibitor of IKKα, IKKβ, IKKε, IκB kinase, TBK1, PKD1, NF-κB, Akt, PKR, TAK1, IRAK1/4 or proteasome.

In some embodiments, the agent is able to:1) inhibit the phosphorylation of IκBα; 2) inhibit the function of IκB kinase; 3) inhibit the function of Akt; or 4) inhibit the function of NF-κB, p38 and JNK signaling.

In some embodiments, the agent is selected from the group consisting of BX795, BAY11-7082, Curcumin, Dexamethasone, 2-Aminopurine, (5Z)-7-Oxozeaenol, IRAK1/4 Inhibitor I, and Bortezomib.

In some embodiments, the viral vector is a VSV-G pseudotyped lentiviral vector.

In some embodiments, the agent capable of inhibiting the innate anti-virus activity of the γδ T cells is BX795.

In some embodiments, BX795 is used at a concentration between 0.02 μM-60 μM, more preferably 0.2 μM-6 μM, and most preferably 0.4 μM-2 μM.

In some embodiments, BX795 is used at a concentration no more than 2 μM.

In some embodiments, BX795 is used at a concentration between 0.2-0.6 μM.

In some embodiments, BAY11-7082 is used at a concentration between 0.1 μM-2000 μM, more preferably 0.5 μM-200 μM, and most preferably 5 μM-100 μM; or BAY11-7082 is used at a concentration between 0.5 μM-50 μM and more preferably 5 μM-50 μM.

In some embodiments, Curcumin is used at a concentration between 0.1 μM-500 μM, more preferably 1 μM-100 μM, and most preferably 2 μM-20 μM; or Curcumin is used at a concentration between 1 μM-100 μM and more preferably 10 μM-100 μM or 1 μM-10 μM.

In some embodiments, Dexamethasone is used at a concentration between 0.01 μM-500 μM, more preferably 0.1 μM-50 μM, and most preferably 1 μM-10 μM; or Dexamethasone is used at a concentration between 0.064 μM-6.4 μM and more preferably 0.64 μM-6.4 μM.

In some embodiments, 2-Aminopurine is used at a concentration between 0.5 μM-5000 μM, more preferably 5 μM-1000 μM, and most preferably 50 μM-500 μM; or 2-Aminopurine is used at a concentration between 5 μM-500 μM and more preferably 50 μM-500 μM.

In some embodiments, (5Z)-7-Oxozeaenol is used at a concentration between 0.01 μM-600 μM, more preferably 0.6 μM-60 μM, and most preferably 0.6 μM-6 μM; or (5Z)-7-Oxozeaenol is used at a concentration between 0.6 μM-60 μM and more preferably 0.6 μM-6 μM.

In some embodiments, IRAK1/4 Inhibitor I is used at a concentration between 0.01 μM-300 μM, more preferably 0.03 μM-30 μM, and most preferably 0.3 μM-3 μM; or IRAK1/4 Inhibitor I is used at a concentration between 0.03 μM-3 μM and more preferably 0.3 μM-3 μM.

In some embodiments, Bortezomib is used at a concentration between 0.002 μM-40 μM, more preferably 0.01 μM-4 μM, and most preferably 0.01 μM-0.4 μM; or Bortezomib is used at a concentration between 0.04 μM-4 μM, such as 0.04 μM.

In another aspect, the present disclosure provides a preparation comprising CAR-γδ T cells prepared by the method described above.

In some embodiments, the CAR-γδ T cells express a CAR comprising an antigen-binding domain targeting to CD4 or B7H3.

In another aspect, the present disclosure provides a pharmaceutical composition for use in treating a tumor comprising the preparation, and a pharmaceutically acceptable carrier.

In some embodiments, the tumor is prostate tumor, T cell leukemia or ovarian cancer.

In another aspect, the present disclosure provides a method for treating a tumor in a subject comprising administrating to the subject a therapeutically effective amount of the preparation or a therapeutically effective amount of the pharmaceutical composition.

In some embodiments, the tumor is prostate tumor, T cell leukemia or ovarian cancer.

The method of transducing γδ T cells provided herein can increase transduction rate and/or prevent the decrease of transduction rate during the subsequent cell expansion process. The method can be used to prepare CAR-γδ T cells for tumor therapy. Without the use of these small molecule inhibitors, the positive rate of CAR-γδ T is quite low which would inhibit its application in clinical application: to get enough CAR positive γδ T cells, more cells should be prepared and more cells are needed to be infused into patients, which would bring more cost of manufacture and more operative risk.

### BRIEF DESCRIPTION OF THE DRAWINGS

FIG. 1 revealed the lentivirus transduction efficiency of conventional up T cells from two donors. The transduction treatment was applied after up T cells were stimulated in vitro for 48 hours. We also calculated the change of the transduction rate during the cell culture progress as long as 16 days.

FIG. 2 contained 4 graphs which revealed the lentivirus transduction of γδ2 T cell with or without 2 μM/6 μM BX795. FIG. 2A showed the total alive cell number during the culture progress, we monitored the data each two days from Day 4 to Day 22. FIG. 2B showed the γδ2 T cell percentage of the total cells during the cell culture time from Day 4 to Day 22. FIG. 2C showed the transduction efficiency of γδ2 T cells and FIG. 2D showed the cell number of positive transduced γδ2 T cells during the cell culture time from Day 4 to Day 22.

FIG. 3 contained 4 graphs which revealed lentivirus transduction of γδ2 T cell with or without BX795 at different concentrations (0.2 μM, 0.6 μM or 2 μM). FIG. 3A showed the total alive cell number during the culture progress, we monitored the data each two days from Day 5 to Day 15. FIG. 3B showed the γδ2 T cell percentage of the total cells during the cell culture time from Day 5 to Day 15. FIG. 3C showed the transduction efficiency of γδ2 T cells and FIG. 3D showed the cell number of positive transduced γδ2 T cells during the cell culture time from Day 5 to Day 15.

FIG. 4 revealed the cytotoxicity of γδ2 T cells to a human prostate tumor cell (PC3). The γδ2 T cells were cultured with or without 0.2 μM or 0.6 μM BX795. The ratio of γδ2 T cells to tumor cells was 3:1 and the cell mix was incubated in normal cell culture condition for 24 hours before analysis of the cytotoxicity efficiency.

FIG. 5 showed the results of the transduction of γδ2 T cells in the presence or absence of 0.6 μM BX-975. (A) transduction rates on D5, D8 and D10; (B) alive cell numbers on D5, D8 and D10.

FIG. 6 showed the results of the transduction of γδ2 T cells in the presence or absence of BAY11-7082 (0.5 μM, 5 μM or 50 μM). (A) transduction rates on D5, D8 and D10; (B) alive cell numbers on D5, D8 and D10.

FIG. 7 showed the results of the transduction of γδ2 T cells in the presence or absence of Curcumin (1 μM, 10 μM or 100 μM). (A) transduction rates on D5, D8 and D10; (B) alive cell numbers on D5, D8 and D10.

FIG. 8 showed the results of the transduction of γδ2 T cells in the presence or absence of Dexamethasone (0.064 μM, 0.64 μM or 6.4 μM). (A) transduction rates on D5, D8 and D10; (B) alive cell numbers on D5, D8 and D10.

FIG. 9 showed the results of the transduction of γδ2 T cells in the presence or absence of 2-Aminopurine (5 μM, 50 μM or 500 μM). (A) transduction rates on D5, D8 and D10; (B) alive cell numbers on D5, D8 and D10.

FIG. 10 showed the results of the transduction of γδ2 T cells in the presence or absence of (5Z)-7-Oxozeaenol (0.6 μM, 6 μM or 60 μM). (A) transduction rates on D5, D8 and D10; (B) alive cell numbers on D5, D8 and D10.

FIG. 11 showed the results of the transduction of γδ2 T cells in the presence or absence of IRAK1/4 Inhibitor I (0.03 μM, 0.3 μM or 3 μM). (A) transduction rates on D5, D8 and D10; (B) alive cell numbers on D5, D8 and D10.

FIG. 12 showed the results of the transduction of γδ2 T cells in the presence or absence of Bortezomib (0.04 μM, 0.4 μM or 4 μM). (A) transduction rates on D5, D8 and D10; (B) alive cell numbers on D5, D8 and D10.

FIG. 13 showed the results of the transduction of γδ1 T cells in the presence or absence of small inhibitors under different dosage including BX795 (0.06 μM, 0.6 μM or 6 μM), BAY11-7082 (0.5 μM, 5 μM or 50 μM), Curcumin (1 μM, 10 μM or 100 μM), Dexamethasone (0.064 μM, 0.64 μM or 6.4 μM), 2-Aminopurine (5 μM, 50 μM or 500 μM), (5Z)-7-Oxozeaenol (0.6 μM, 6 μM or 60 μM), IRAK1/4 Inhibitor I (0.03 μM, 0.3 μM or 3 μM) and Bortezomib (0.04 μM, 0.4 μM or 4 μM).

FIG. 14 showed the killing activity of CAR γδ2 T cells on CD4 positive tumor cells. (A) cytotoxicity to CD4 positive tumor cells; (B) secreted IFNγ; (C) secreted TNFα.

FIG. 15 showed the tumor inhibition activity CAR γδ2 T cells on Jurkat T-luc tumor cells in vivo. (A) bioluminescence imaging photos taken on indicated days; (B) changes of total bioluminescence intensity; (C) survival curves.

FIG. 16 showed the tumor inhibition activity CAR γδ2 T cells on SKOV3-luc tumor cells in vivo. (A) bioluminescence imaging photos taken on indicated days; (B) changes of total bioluminescence intensity.

### DETAILED DESCRIPTION

Unless defined otherwise, technical and scientific terms used herein have the same meaning as commonly understood by a person of ordinary skill in the art. Any methods, devices and materials similar or equivalent to those described herein can be used in the practice of the present invention. The following definitions are provided to facilitate understanding of certain terms used herein and are not meant to limit the scope of the present disclosure.

The articles “a” and “an” are used herein to refer to one or to more than one (i.e., to at least one) of the grammatical object of the article. By way of example, “an element” means one element or more than one element.

Unless the context requires otherwise, the word “comprise” and variations such as “comprises” and “comprising” will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having.”

Unless otherwise stated, any numerical value, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1 mg/mL to 10 mg/mL includes 0.9 mg/mL to 11 mg/mL.

As used herein, the use of a numerical range expressly includes all possible subranges, all individual numerical values within that range, including integers within such ranges and fractions of the values unless the context clearly indicates otherwise.

The term “innate anti-virus activity” as used herein refers to the activity of the innate immune system of a host cell to repress the replication of viruses and/or expression of genes of viruses in the host cell. It is well known in the art that dsRNA or dsDNA censors (e.g., retinoic acid-inducible gene I (RIG-I), cyclic GMP-AMP synthase) in the cytosol can recognize viral nucleic acids and trigger the host cell into an anti-viral state by inducing type I interferon response. “An agent capable of inhibiting the innate anti-virus activity” thus refers to an inhibitor that can prevent the development of the anti-viral state in the host. In a non-limiting example, the agent is an inhibitor of IkB kinase (IKKε) and/or TANK-binding kinase 1 (TBK1), e.g., BX795. In another non-limiting example, inhibitors such as BAY11-7082, Curcumin, Dexamethasone, 2-Aminopurine, (5Z)-7-Oxozeaenol, IRAK1/4 Inhibitor I, and Bortezomib may be used to inhibit the innate anti-virus activity.

The term “vector” as used herein refers to a nucleic acid construct or sequence, generated recombinantly or synthetically, with specific nucleic acid elements that permit transcription and/or expression of another foreign or heterologous nucleic acid in a host cell. A vector can be a plasmid, virus, or nucleic acid fragment. A vector can include nucleic acid sequences that permit it to replicate in a host cell, such as an origin of replication. A vector can also include one or more selectable marker genes and other genetic elements. The vector can be an expression vector which contains the necessary regulatory sequences to allow transcription and/or translation of an inserted target gene or genes. In some non-limiting examples, the vector is a viral vector, such as a lentiviral vector. Viral vectors suitable for gene delivery to γδ T cells include, for example, retrovirus, adenovirus, adeno-associated virus, vaccinia virus, and lentivirus vectors. In non-limiting examples disclosed herein, γδ T cells are transduced with lentiviral vectors including one or more heterologous nucleic acids encoding one or more target proteins (e.g., GFP or CAR).

The term “transduce”, “transducing” or “transduction” refers to transferring nucleic acid into a host cell, such as transfer of a heterologous nucleic acid into a host cell. As used herein, the term includes all techniques by which a nucleic acid is introduced into a cell, including but not limited to transformation with plasmid vectors, infection with viral vectors or viral particles, and introduction of naked DNA by electroporation, nucleofection, lipofection, or particle gun.

The term “pseudotyping” or “pseudotyped” as used herein refers to a vector particle bearing envelope glycoproteins derived from other viruses having envelopes. In a non-limiting example, the lentiviral vector used to transduce γδ T cells is a VSV-G pseudotyped lentiviral vector.

The term “chimeric antigen receptor (CAR)” as used herein refers to an artificial receptor protein, which is intended to be expressed on the surfaces of immune cells, particularly T cells, and give the immune cells a new ability to target specific antigens (e.g., tumor specific antigens) on target cells (e.g., tumor cells). The receptors are “chimeric” because they combine both antigen-binding and T-cell activating functions into a single receptor. In their usual format, chimeric antigen receptors graft the specificity of a monoclonal antibody (mAb) to the effector function of a T cell. Once the CARs are expressed in a T cell, the CAR modified T cell (CAR-T or CAR-T cell) acquires some properties, such as antigen specific recognition, antitumor reactivity and proliferation, and thus can act as “living drugs” to eradicate targeted tumor cells. CAR-T cell therapy can override tolerance to self-antigens and provide a treatment which is not reliant on the MHC status of a patient.

CARs are expressed as transmembrane proteins, including an antigen-specific binding site, a transmembrane region, and a signaling cytoplasmic domain (e.g., a CD3(chain). The antigen-specific binding site is usually a monoclonal antibody-derived single chain variable fragment (scFv) consisting of a heavy and light chain joined by a flexible linker. Recently CAR constructs have incorporated additional cytoplasmic domains from co-stimulatory molecules such as CD28 or 4-1 BB to enhance T cell survival in vivo. A CAR may comprise an extracellular domain, a transmembrane domain and an intracellular domain. In some embodiments, the CAR further includes a signal peptide at N-terminus, and a hinge region between the extracellular domain and the transmembrane domain. The extracellular domain includes a target-specific binding element (also referred to as an antigen recognition domain or antigen binding domain). The intracellular domain, or otherwise the cytoplasmic domain, often includes one or more co-stimulatory signaling domains and a CD3 (chain portion. The co-stimulatory signaling domain refers to a portion of the CAR including the intracellular domain of a co-stimulatory molecule. Antigen recognition or antigen targeting by a CAR molecule most commonly involves the use of an antibody or antibody fragment. In some embodiments, the antigen binding domain is an antibody or antibody fragment that specifically binds to CD4 or B7H3.

The term “NF-κB signaling pathway” as used herein refers to a signaling pathway leading to the activation or deactivation of a NF-κB transcription factor. NF-κB transcription factors are critical regulators of immunity, stress responses, apoptosis and differentiation. In mammals, there are five members of the transcription factor NF-κB family: RELA (p65), RELB and c-REL, and the precursor proteins NF-κB1 (p105) and NF-κB2 (p100).

NF-κB transcription factors bind as dimers to KB sites in promoters and enhancers of a variety of genes and induce or repress transcription. NF-κB activation occurs via two major signaling pathways: the canonical and the non-canonical NF-κB signaling pathways. The canonical NF-κB pathway is triggered by signals from a large variety of immune receptors, such as TNFR, TLR, and IL-1R, which activate TAK1. TAK1 then activates IκB kinase (IKK) complex, composed of catalytic (IKKα and IKKβ) and regulatory (NEMO) subunits, via phosphorylation of IKKβ. Upon stimulation, the IKK complex, largely through IKKβ, phosphorylates members of the inhibitor of KB (IκB) family, such as IκBa and the IκB-like molecule p105, which sequester NF-κB members in the cytoplasm. IκBa associates with dimers of p50 and members of the REL family (RELA or c-REL), whereas p105 associates with p50 or REL (RELA or c-REL). Upon phosphorylation by IKK, IκBa and p105 are degraded in the proteasome, resulting in the nuclear translocation of canonical NF-κB family members, which bind to specific DNA elements, in the form of various dimeric complexes, including RELA-p50, c-REL-p50, and p50-p50. Atypical, IKK-independent pathways of NF-κB induction also provide mechanisms to integrate parallel signaling pathways to increase NF-κB activity, such as hypoxia, UV and genotoxic stress. The non-canonical NF-κB pathway is induced by certain TNF superfamily members, such as CD40L, BAFF and lymphotoxin-β (LT-β), which stimulates the recruitment of TRAF2, TRAF3, cIAP1/2 to the receptor complex. Activated cIAP mediates K48 ubiquitylation and proteasomal degradation of TRAF3, resulting in stabilization and accumulation of the NF-κB-inducing kinase (NIK). NIK phosphorylates and activates IKKα, which in turn phosphorylates p100, triggering p100 processing, and leading to the generation of p52 and the nuclear translocation of p52 and RELB.

The term “pharmaceutical composition” refers to a preparation comprising an active ingredient and a physiologically acceptable excipient that is in such form as to permit the biological activity of the active ingredient to be effective. As used herein, “physiologically acceptable excipient” includes without limitation any adjuvant, carrier, diluent, preservative, dispersing agent, suspending agent, stabilizer, isotonic agent, solvent, surfactant, or emulsifier as being acceptable for use in humans or domestic animals. In some embodiments, the CAR-T cells of the present invention or the pharmaceutical composition comprising the same is used to treat a tumor (or cancer) in a subject.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of the disease. The methods of the invention contemplate any one or more of these aspects of treatment.

The term “therapeutically effective amount” may include an amount that is effective to “treat” a subject.

When a therapeutic amount is indicated, the precise amount contemplated in particular embodiments, to be administered, can be determined by a physician in view of the condition of the subject.

As used herein, the term “subject” refers to an organism to which the CAR γδ T cells or a composition comprising CAR γδ T cells of the present invention is to be administered. Preferably, a subject is a mammal, e.g., a human.

As used herein, the term “preparation” refers to a product or manufacture comprising the CAR γδ T cells prepared by the method of the present invention. As nonlimiting examples, the preparation may be in a form of solution, suspension, etc.

BX795 is an inhibitor of TANK-binding kinase 1 (TBK1) and IκB kinase ε (IKKε). Its formula is as follows (CAS Accession Number: 702675-74-9): Other inhibitors used in the present invention have formulas as follows:

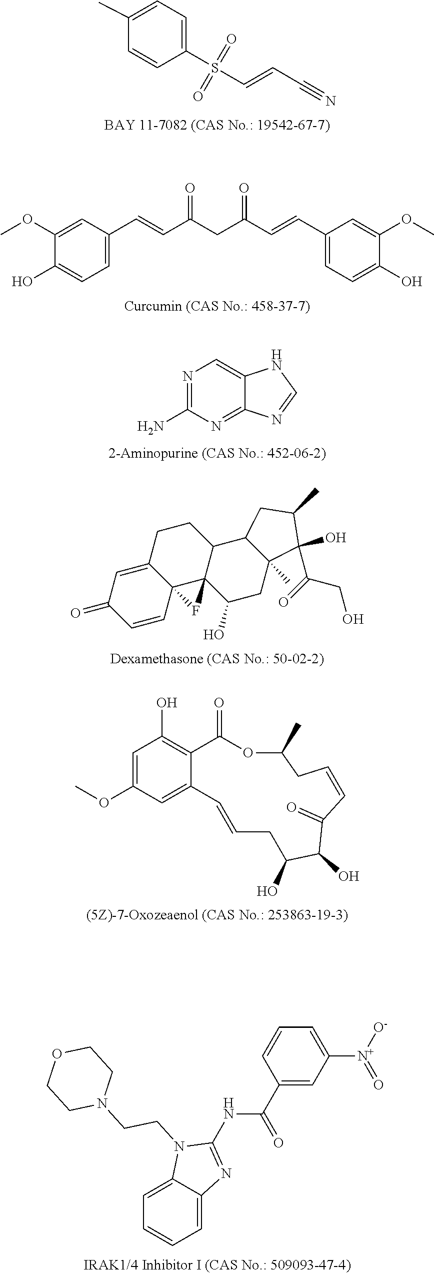

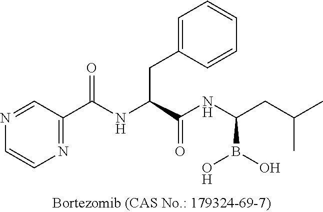 [SMILES: CC(C)C[C@H](NC(=O)[C@H](Cc1ccccc1)NC(=O)c1cnccn1)B(O)O]

The inventors of the present invention find that when γδ T cells are transduced with viral vectors, the transduction rate may decrease significantly during 4-8 days after the transduction. Generally, the viral vectors contain at least a target gene to be expressed in host cells. Thus the change of the transduction rate can be monitored by measuring the percentage of positive cells (i.e., cells expressing the target gene) through flow cytometry.

The inventors of the present invention unexpectedly find that when γδ T cells are transduced with viral vectors in the presence of an agent capable of inhibiting the innate anti-virus activity (hereinafter referred to as “innate anti-virus activity inhibitor”) of the γδ T cell, such as BX795, the transferred viral vectors can stably remain in the γδ T cells, even though the γδ T cells are thereafter cultured in a medium without supplement of the innate anti-virus activity inhibitor (e.g., BX795). The maintenance of the vectors in the cells can also be detected by, such as, flow cytometry. This is critical for CAR-γδ T cells if they are to be returned to patients for tumor treatment. Before the treatment, we need to prepare a sufficient number of CAR-positive γδ T cells. If the positive rate gradually decreases during in vitro expansion of γδ T cells, it is impossible to obtain a sufficient amount of positive cells for clinical application. Moreover, the continued decline in the positive rate indicates that the cells after reinfusion may lose CARs in vivo, thus losing the therapeutic effect.

The inventors of the present invention further find that when the inhibitor (e.g., BX795) is used in a suitable concentration, it will not impair cell growth and expansion of the γδ T cells while improving and/or maintaining the transduction rate. In some embodiments, BX795 is used at a concentration of 0.02 μM-60 μM, more preferably 0.2 μM-6 μM, and most preferably 0.4 μM-2 μM. In other embodiments, BX795 is used at a concentration of 0.2 μM-6 μM, such as 0.2 μM-0.6 μM. In some embodiments, BX795 is used in a concentration of no more than 2 μM, such as 0.2 μM-2 μM. In some preferred embodiments, BX795 is used at a concentration of 0.2 μM-0.6 μM, such as 0.3, 0.4, 0.5 or 0.6 μM. In a more preferred embodiment, BX795 is used in a concentration of 0.6 μM. In some embodiments, BAY11-7082 is used at a concentration between 0.1 μM-2000 μM, more preferably 0.5 μM-200 μM, and most preferably 5 μM-100 μM. In other embodiments, BAY11-7082 is used at a concentration of 0.5 μM-50 μM, such as 5 μM-50 μM. In non-limiting examples, BAY11-7082 is used at a concentration of 1, 2, 3, 4 5, 6, 7, 8, 9, 10, 20, 30, 40, or 50 μM. In some embodiments, Curcumin is used at a concentration of 0.1 μM-500 μM, more preferably 1 μM-100 μM, and most preferably 2 μM-20 μM. In other embodiments, Curcumin is used at a concentration of 1 μM 100 μM, such as 10 μM-100 μM or 1 μM-10 μM. In non-limiting examples, Curcumin is used at a concentration of 1, 2, 3, 4 5, 6, 7, 8, 9, 10, 20, 30, 40, 50, 60, 70, 80, 90, or 100 μM. In some embodiments, Dexamethasone is used at a concentration of 0.01 μM-500 μM, more preferably 0.1 μM-50 μM, and most preferably 1 μM-10 μM. In other embodiments, Dexamethasone is used at a concentration of 0.064 μM-6.4 μM, such as 0.64 μM-6.4 μM. In non-limiting examples, Dexamethasone is used at a concentration of 0.1, 0.2, 0.3, 0.4 0.5, 0.6, 0.7, 0.8, 0.9, 1, 2, 3, 4, 5, or 6 μM. In some embodiments, 2-Aminopurine is used at a concentration of 0.5 μM-5000 μM, more preferably 5 μM-1000 μM, and most preferably 50 μM-500 μM. In other embodiments, 2-Aminopurine is used at a concentration of 5 μM-500 μM, such as 50 μM-500 μM. In non-limiting examples, 2-Aminopurine is used at a concentration of 10, 20, 30, 40, 50, 60, 70, 80, 90, 100, 200, 300, 400, or 500 μM. In some embodiments, (5Z)-7-Oxozeaenol is used at a concentration of 0.01 μM-600 μM, more preferably 0.6 μM-60 μM, and most preferably 0.6 μM-6 μM. In other embodiments, (5Z)-7-Oxozeaenol is used at a concentration of 0.6 μM-60 μM, such as 0.6 μM-6 μM. In non-limiting examples, (5Z)-7-Oxozeaenol is used at a concentration of 0.7, 0.8, 0.9, 1.0, 1.1, 1.2, 1.3, 1.4, 1.5, 1.6, 1.7, 1.8, 1.9, 2.0, 3.0, 4.0, 5.0, or 6.0 μM. In some embodiments, IRAK1/4 Inhibitor I is used at a concentration of 0.01 μM-300 μM, more preferably 0.03 μM-30 μM, and most preferably 0.3 μM-3 μM. In other embodiments, IRAK1/4 Inhibitor I is used at a concentration of 0.03 μM-3 μM, such as 0.3 μM-3 μM. In non-limiting examples, IRAK1/4 Inhibitor I is used at a concentration of 0.05, 0.08, 0.1, 0.5, 0.8, 1.0, 1.2, 1.6, 1.8, 2.0, 2.3, 2.5 or 3.0 μM. In some embodiments, Bortezomib is used at a concentration of 0.002 μM-40 μM, more preferably 0.01 μM-4 μM, and most preferably 0.01 μM-0.4 μM. In other embodiments, Bortezomib is used at a concentration of 0.04 μM-4 μM, such as 0.04 μM. A concentration beyond the ranges described above may also be used with the present invention, provided that the inhibitor of this concentration is able to improve the transduction rate (increasing and/or maintaining the transduction rate) and will not significantly impair cell growth and expansion of the γδ T cells.

Accordingly, the present disclosure provides a method for transducing a γδ T cell with a viral vector in the present of an innate anti-virus activity inhibitor (e.g., BX795). The use of the inhibitor can improve the transduction rate and prevent the loss of the viral vector after the transduction process. The present disclosure also provides a method for preparing CAR-γδ T cells, which comprises transducing a γδ T cell with a viral vector comprising a nucleotide sequence encoding a chimeric antigen receptor in the present of an innate anti-virus activity inhibitor (e.g., BX795). The use of the innate anti-virus activity inhibitor (e.g., BX795) will not unfavorably influence viability and killing activity of γδ T cells or CAR-γδ T cells.

### Examples

The main goal of this invention is to stabilize and improve the virus transduction efficiency of γδ T cells, which could further be applied to construct the chimeric antigen receptors expressing γδ T cells (CAR-γδ T cells). According to the data we have got, in the absence of anti-virus inhibitors, the virus transduction efficiency of the up T cells was very high which was around 60% and the transduction rate remained stable at least for 2 weeks during the in vitro culture condition. For γδ T cells, however, the transduction rate decreased sharply from 80% to 20% from day 4 (48 hours after virus transduction) to day 8 of the in vitro culture. Adding BX795 (the final concentration was 0.6 μM) could inhibit the decrease of transduction rate and the final transduction efficiency could be remained at 65%. On the other hand, BX795 had no damage to γδ T cells, and the harvested γδ T cells could be used to perform subsequent functional experiments. Experimental results obtained with other small inhibitors were also provided. Thus, our invention resolved the problem of the decrease of transduction rate in virus transduction of γδ T cells, which could be further used for gene editing of γδ T cells such as developing the CAR-γδ T cells.

### Cell Lines

293T cells and SKOV3 cells were maintained in Dulbecco's Modified Eagle Medium (DMEM) (Gibco) supplemented with 10% Fetal Bovine Serum (FBS) (GIBCO), 0.1 mM non-essential amino acids and 6 mM L-glutamine.

Jurkat T cells were maintained in RPMI-1640 medium (Gibco) supplemented with 10% Fetal Bovine Serum (FBS) (GIBCO), 0.1 mM non-essential amino acids and 6 mM L-glutamine.

### Production of Lentiviral Vectors

VSV-G pseudotyped lentiviral vectors were applied in this method. 1×10\hat{ }7 293T cells were plated into a poly-D-lysine coated 100 mm dish. Next day the cells were transfected with 6 μg of pCDH-EF1-MCS-T2A-copGFP plasmid (Addgene, Plasmid #72263) or pCDH-EF1-CAR-T2A-copGFP plasmid modified from pCDH-EF1-MCS-T2A-copGFP, 4 μg of pspAx2 (Addgene, Plasmid #12260), 2 μg of pCMV-VSV-G (Addgene, Plasmid #8454) using 30 ug PEI transfection regents. After 8 hours of transfection, the cell culture medium was changed. The supernatant were collected 48 hours and 72 hours later. Concentrated the virus with Lenti-X™ Concentrator (Takara) and monitored the virus titers by transduction of 293T cells and stored the concentrated virus in −80° C. until further use.

### Primary Cell Culture

The peripheral blood mononuclear cells (PBMCs) were isolated by gradient centrifugation, using Ficoll-Paque Plus (GE Healthcare) and washed twice with phosphate-buffered saline (PBS). Cell count and viability were assessed by AO/PI staining. For γδ2 T cells amplification: PBMCs were cultured in serum free medium (Gibco) at the concentration of 2×10\hat{ }cells/ml, and supplemented with 1000 U/ml rhIL-2 and 5 μM ZOL. For γδ1 T cells amplification: PBMCs were cultured in serum free medium (Gibco) at the concentration of 1×10\hat{ }cells/ml in culture plate pre-coated with purified TS-1 monoclonal antibody (NOVUS, NBP2-22488), and supplemented with 1000 U/ml rhIL-2. For conventional αβT cells amplification, PBMCs were cultured in serum free medium at the concentration of 2×10\hat{ }cells/ml in culture plate pre-coated with purified anti-human CD3 and anti-human CD28 monoclonal antibodies, and supplemented with 1000 U/ml rhIL-2.

### Lentiviral Transduction of αβ or γδ T Cells

For lentivirus transduction, 1×10\hat{ }CFU lentivirus diluted in 200 ul PBS were added in a 24-well plate which were pre-coated with RetroNectin reagent (Takara) and centrifugated by 2,000 g for 2 hours at 32° C. After centrifugation, removed the supernatant and washed the plate with PBS three times slightly.

For the virus transduction of the up T cells, seeded 1×10\hat{ }PBMCs into RetroNectin reagent pre-coated plate which were stimulated by anti-human CD3/CD28 monoclonal antibodies for 48 hours in vitro.

Concentrate the cells by 800 g for 10 mins at 32° C. The plates were incubated at 37° C., 5% CO₂.

For the virus transduction of γδ2 T cells, seeded 1×10\hat{ }PBMCs which were in vitro cultured after 48 hours in the γδ2 T cell culture medium mentioned above (Gibco serum free medium with rhIL-2 and ZOL). Added or not small inhibitors and mixed well and concentrated the cells by 800 g for 10 mins at 32° C. The plates were incubated at 37° C., 5% CO₂. Discarded the small inhibitors regent by changing the cell culture medium 24 hours later.

For the virus transduction of γδ1 T cells, seeded 1×10\hat{ }PBMCs which were in vitro cultured after 48 hours in the γδ1 T cell culture medium mentioned above (Gibco serum free medium with rhIL-2 and PBMC were pre-stimulated by TS-1 monoclonal antibody). Added or not small inhibitors and mixed well and concentrated the cells by 800 g for 10 mins at 32° C. The plates were incubated at 37° C., 5% CO₂. Discarded the small inhibitors regent by changing the cell culture medium 24 hours later.

Calculate the cell number by an automated cell counter and the transduction rate (GFP positive rate) was analyzed by flow cytometry every 2 to 3 days. The transduction rate was monitored in the gate of γδ2 or γδ1 T cells.

### Flow Cytometry

Wash the cells once with PBS and then staining the cells with antibodies diluted in FACS buffer (PBS+1% FBS+2.5 mM EDTA) at 4° C. for 30 min. The common volume of incubated buffer was 50 μL for 2×10\hat{ }cells.

After incubation, washed the cells with FACS buffer two times and then resuspended the cells in 200 ul FACS buffer and calculated the data by FACSCalibur (BD Biosciences). The antibodies used for γδ2 T cells were: APC anti-human CD3 (Biolegend, 300412), BV421 anti-human TCR Vδ2 (Biolegend, 331428).

### Cytotoxicity Assay In Vitro

Resuspend the effector T cells and tumor cells which stably expressed firefly luciferase with fresh serum free medium (Gibco). Modified the cell density and seed the effector T cells and tumor cells in 96 well plates at different ratio effector T cells to tumor cells. The final volume of each well is 100 ul and the cell number of tumor cells is 10 thousand.

Culture the cell mix in 37° C., 5% CO₂ for 12 hours and mix the cells completely, take 50 ul cells into another 96 well plate and add the luciferase substrate follow the instruction of the kit (Luciferase Assay System, Promega, Cat: E1500). Read the plate by Luminometers.

### Mouse Experiments

For in vivo efficacy studies, 7 to 9-week-old female NOD.Cg-Prkdcscid IL2rgtm1Wjl/SzJ (NSG) mice were implanted by tail intravenous injection (i.v.) with 1×10⁶ Jurkat T or intraperitoneal injection (i.p.) 1×10⁶ SKOV3 cells. Both Jurkat T and SKOV3 cell were stably express firefly luciferase (day 0). 5×10⁶γδ T cells were injected into the tumor bearing mice at day 5, day 8, day 11, day 14 and day 17 for Jurkat T CDX model (i.v.) and 5×10⁶γδ T cells were injected into the tumor bearing mice at day 5, day 8 and day 11 for SKOV3 CDX model (i.p.). Tumor volume was measured by IVIS Lumina LT system (PerkinElmer).

### Example 1. Lentivirus Transduction Efficiency of the Conventional T Cells (Up T Cells)

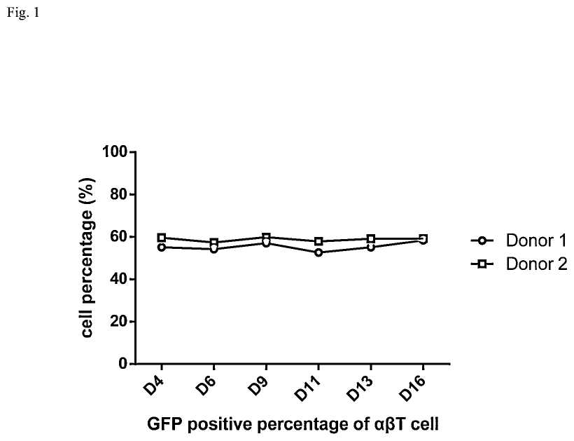

The lentivirus transduction of the conventional T cells was applied on Day 2 (48 hours later of the in vitro culture). The transduction efficiency was monitored every 2 or 3 days from Day 4 to Day 16 (FIG. 1). It can be seen from FIG. 1, the transduction rate was around 60% and remained stable in the whole culture progress.

The T cells were obtained from two different donors.

### Example 2. Lentivirus Transduction Efficiency of 762 T Cells could be Improved by BX795 and High Dosage of BX795 Impaired the Cell Growth of 762 T Cell

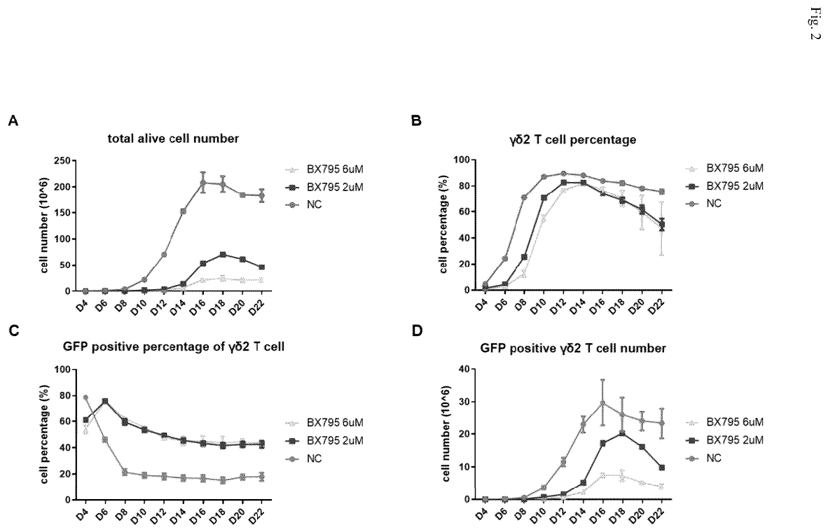

The lentivirus transduction of γδ2 T cells was applied on Day 2 (48 hours later of the in vitro culture). The transduction efficiency was monitored each two days until Day 22 and the total cell number and γδ2 T cell percentage were calculated either (FIG. 2). It can be seen from FIGS. 2A and 2B, 2μM or 6 μM BX795 impaired the cell growth of γδ2 T cells, that the total alive cell number (FIG. 2A) and γδ2 T cell percentage (FIG. 2B) were dramatically lower than the control group without BX795.

On the other hand, for control group, the virus transduction rate decreased sharply from 80% to 20% from Day 4 to Day 8, and then remained stable (FIG. 2C). As BX795 added during the transduction progress, the virus transduction efficiency finally remained at ˜40% which was significantly higher than the control group (FIG. 2C). Though at higher transduction rate of the BX795 application groups, the cell number of transduced γδ2 T cells was lower than the control group (FIG. 2D) which was caused by the cell growth inhibition of BX795 at high dosage.

Thus, BX795 application in the lentivirus transduction progress could enhance the transduction efficiency but inhibit the cell growth of γδ2 T cells. Decreased the BX795 dosage may improve the transduction efficiency but with no influence on γδ2 T cell growth.

### Example 3. Low Dosage of BX795 Improved the Lentivirus Transduction of 762 T Cells without Influencing the Cell Growth of 762 T Cells

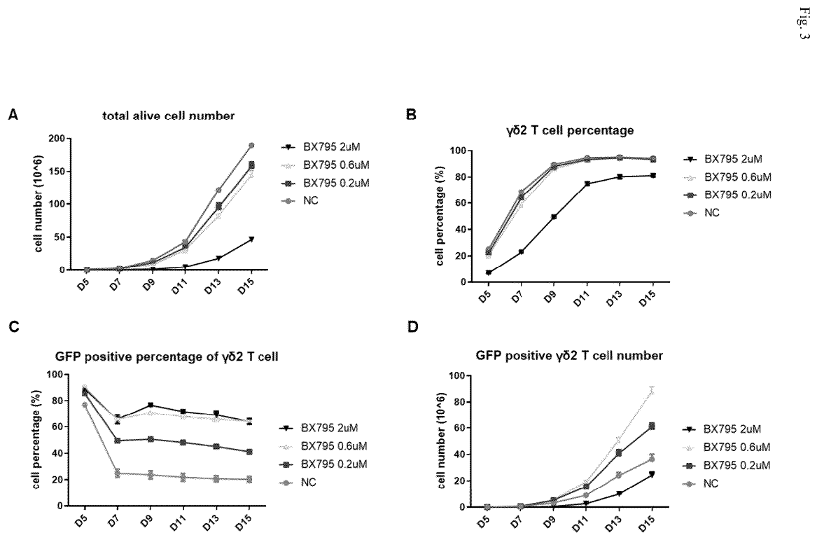

To further study the usage of BX795 on lentivirus transduction of γδ2 T cells, we compared the function of BX795 from 0.2 μM to 2 μM (FIG. 3). 2 μM BX795 significantly decreased the total cell number and γδ2 T cell percentage compared with the control group (FIGS. 3A and 3B). However, under low dosage of BX795 (0.2 μM or 0.6 μM), γδ2 T cell growth was not significantly affected.

On the concern of transduction efficiency, 0.2 μM BX795 enhanced the transduction efficiency from around 20% to above 40%, and 0.6 μM BX795 led to the final transduction rate reached to around 65% which was not significant with 2 μM BX795 (FIG. 3C). The total positive transduced γδ2 T cells were also greatly increased as 0.2 μM or 0.6 μM BX795 was applied (FIG. 3D).

The data revealed adding 0.2 μM or 0.6 μM BX795 during the lentivirus transduction progress could both help to improve the lentivirus transduction efficiently.

### Example 4. BX795 had No Significant Impact on Cell Cytotoxicity of 762 T Cells

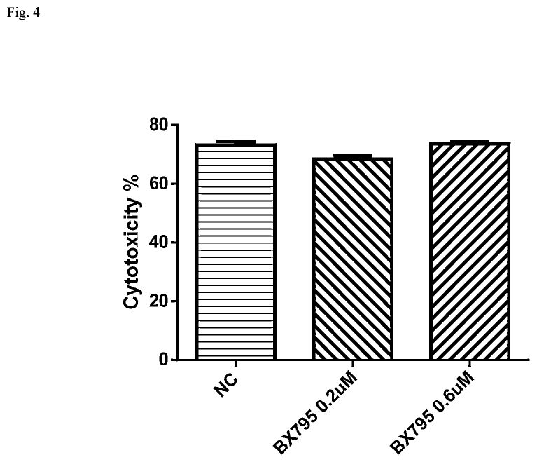

To evaluate whether BX795 could influence the tumor cell killing ability of γδ2 T cells, we cultured γδ2 T cells with 0.2 or 0.6 μM BX795 and tested the cytotoxicity efficiency to PC3 tumor cells (one human prostate tumor cell line) (FIG. 4). The cell number ratio of γδ2 T cells to PC3 cells were 3:1 and the killing time was 24 hours. It can be seen that the control γδ2 T cells cultured without BX795 possessed cell killing efficiency of 73.2%. The cytotoxicity of γδ2 T cells cultured with 0.6 μM and 0.2 μM BX795 was 73.6% and 68.4%, respectively. Thus, BX795 had no significant impact on cell cytotoxicity of γδ2 T cells.

### Example 5. BX795 had No Significant Influence on the Cell Types of the Final γδ T Cell Products Developed from PBMC

We evaluated the cell types of the final γδ T cell products cultured from PBMC (after 12 Days in vitro culture), with or without 0.6 μM BX795. The data was shown in Table 1. The calculated cell types included γδ2 T, γδ2 CD56+ T, γδ1 T, αβT, NKT, T helper, cytotoxic T, B and NK cells. It can be seen BX795 applied culture condition resulted in comparable cell types with the control group.

TABLE 1
Cell types of the final γδ T cell products cultured with/without BX795

| | | Cell percentage of total lymphocytes | | | |
|---|---|---|---|---|---|
| Cell type | Markers | NC | | BX795 | |
| γδ2 T cell | CD3+TCRVδ2+ | 80.22% | 79.73% | 85.93% | 86.52% |
| γδ2 CD56+ T cell | CD3+TCRVδ2+ CD56+ | 2.07% | 1.55% | 1.89% | 1.69% |
| γδ2 T cell | CD3+TCRVδ2−TCRαβ−TCRVδ1+ | 0.83% | 0.73% | 0.51% | 0.38% |
| αβ T cell | CD3+TCRVδ2−TCRVδ1−TCRαβ+ | 10.04% | 10.54% | 7.22% | 7.20% |
| NKT cell | CD3+TCRVδ2−TCRVδ1−TCRαβ+CD56+ | 0.50% | 0.44% | 0.34% | 0.31% |
| T helper cell | CD3+TCRVδ2−TCRVδ1− | 7.29% | 7.75% | 5.26% | 5.40% |
| | TCRαβ+CD56−CD8−CD4+ | | | | |
| Cytotoxic T cell | CD3+TCRVδ2−TCRVδ1− | 2.14% | 2.17% | 1.52% | 1.39% |
| | TCRαβ+CD56−CD8+CD4− | | | | |
| B cell | CO3−TCRVδ2−CD19+ | 0.42% | 0.30% | 0.27% | 0.32% |
| NK cell | CD3−TCRVδ2−CD56+ | 4.52% | 5.11% | 2.66% | 2.20% |

Table 1 revealed the cell types of the final γδ T cell products cultured with or without BX795. This analysis was applied to study the effect of BX795 to the total cell differentiation in the culture progress. Different cell types including γδ2 T, γδ2 CD56+ T, γδ1 T, αβT, NKT, T helper, cytotoxic T, B and NK cells were evaluated.

### Example 6. BX795 had No Significant Influence on the Differentiation of 762 T Cells Developed from PBMC

We compared the differentiation of γδ2 T cell with BX795 treatment, various γδ2 T subtypes including CD226 positive γδ2 T cells, NKG2D positive γδ2 T cells, naïve γδ2 T cells, central memory γδ2 T cells, effector γδ2 T cells and terminator γδ2 T cells were calculated. No significant changes were found (Table 2). For the central memory γδ2 T cells, adding BX795 could improve the percentage rate from 1.865% to 4.225%. On the other hand, the terminator γδ2 T cell percentage decreased from 2.595% to around 2%.

TABLE 2
Differentiation of γδ2 T cells cultured with/without BX795

| | | Cell percentage of total lymphocytes | | | |
|---|---|---|---|---|---|
| Cell type | Markers | NC | | BX795 | |
| γδ2 T cell | CD3+TCRVδ2+ | 80.05% | 79.88% | 87.25% | 86.68% |
| γδ2 CD226+T cell | CD3+TCRVδ2+CD226+ | 79.97% | 79.80% | 87.25% | 86.59% |
| γδ2 NKG2D+T cell | CD3+TCRVδ2+NKG2D+ | 79.89% | 79.72% | 87.17% | 86.50% |
| naive γδ2 T cell | CD3+TCRVδ2+ | 0.10% | 0.08% | 0.25% | 0.10% |
| | CD27+CD45RA+ | | | | |
| Central memory | CD3+TCRVδ2+ | 1.97% | 1.76% | 3.88% | 4.57% |
| γδ2 cell | CD27+CD45RA− | | | | |
| Effector γδ2 T cell | CD3+TCRVδ2+ | 75.41% | 75.41% | 81.23% | 80.00% |
| | CD27−CD45RA+ | | | | |
| Terminator γδ2 T cell | CD3+TCRVδ2+ | 2.55% | 2.64% | 1.91% | 2.03% |
| | CD27−CD45RA+ | | | | |

Table 2 revealed the differentiation of γδ2 T cells cultured with or without BX795. This analysis was applied to study the effect of BX795 to the γδ2 T cell differentiation in the culture progress. Different γδ2 T cell subtypes including CD226+γδ2 T cells, NKG2D+γδ2 T cells, naïve γδ2 T cells, central memory γδ2 T cells, effector γδ2 T cells and terminator γδ2 T cells were evaluated.

### Example 7. BX795 Slightly Increased the Exhausted Gene Expression of 762 T Cell

To calculate whether BX795 influenced the exhaustion of γδ2 T cells, we checked some classical exhausted genes expressed on γδ2 T cells including PD-1, LAG-3, TIGIT and TIM-3 (Table 3). The data revealed BX795 treatment improved the cell percentage of all the exhausted cell types slightly.

TABLE 3
Exhausted cell percentage of γδ2 T cell products cultured with/without BX795

| | | Cell percentage of total lymphocytes | | | |
|---|---|---|---|---|---|
| Cell type | Markers | NC | | BX795 | |
| γδ2 T cell | CD3+TCRVδ2+ | 78.80% | 79.78% | 85.75% | 85.94% |
| γδ2 PD-1+ T cell | CD3+TCRVδ2+PD-1+ | 7.30% | 6.66% | 11.83% | 11.17% |
| γδ2 LAG-3+ T cell | CD3+TCRVδ2+LAG-3+ | 78.66% | 79.62% | 85.58% | 85.77% |
| γδ2 TIGIT+ T cell | CD3+TCRVδ2+TIGIT+ | 17.91% | 16.08% | 23.24% | 26.98% |
| γδ2 TIM-3+ T cell | CD3+TCRVδ2+TIM-3+ | 77.24% | 78.18% | 84.55% | 84.91% |

Table 3 revealed the expression level of exhausted markers of γδ2 T cells cultured with or without BX795. Exhausted genes including PD-1, LAG-3, TIGIT and TIM3 were calculated.

### Example 8. BX795 Improved CAR Related Lentivirus Transduction of 762 T Cell

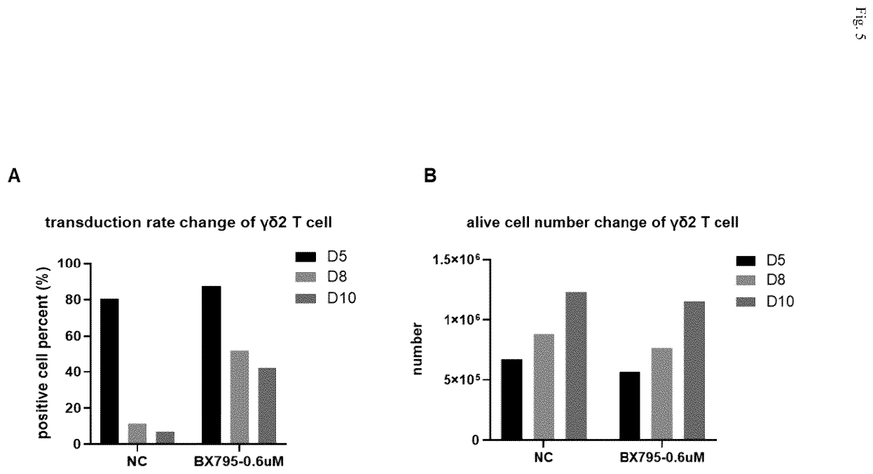

To further test the application of BX795 on lentivirus transduction, we transduced the γδ2 T cell with CAR (including the scFv domain recognizing B7H3 molecule, CD8 hinge/transmembrane, CD28 and CD137 co-stimulatory domain and CD3(activation domain) related lentivirus. As shown in FIG. 5A, the transduction rate of the control group decreased quickly from Day5 to Day8 which was below 10%. With BX795 (0.6 uM), the transduction rate remained stable around 40% at day 10. As the data mentioned above, the adding of BX795 did not influence the cell growth of γδ2 T (FIG. 5B).

### Example 9. BAY11-7082 Improved CAR Related Lentivirus Transduction of 762 T Cell

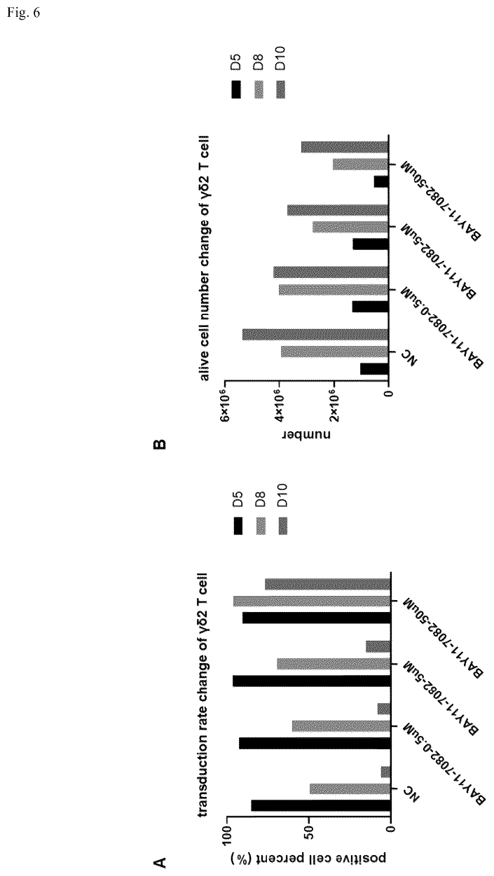

The transduction rate of the control group decreased continuously from Day5 to Day10 which was around 5%. BAY11-7082 could enhance the transduction rate in a dosage dependent manner from 0.5 uM to 50 uM (FIG. 6A). At the dosage of 50 uM, the transduction rate was higher than 70%. The adding of BAY11-7082 impaired the cell growth in a dosage dependent manner either and higher dosage resulted in less total cell number (FIG. 6B).

### Example 10. Curcumin Improved CAR Related Lentivirus Transduction of 762 T Cell

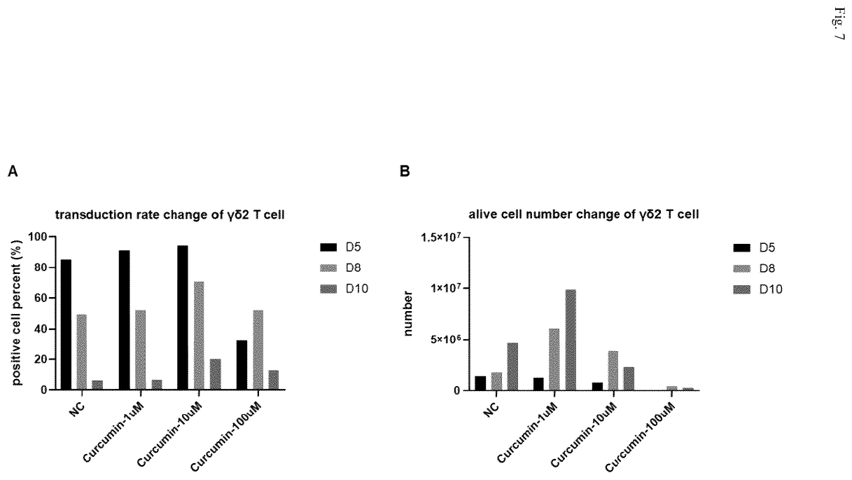

The transduction rate of the control group decreased continuously from Day5 to Day10 which was around 5%. With Curcumin (10 uM), the transduction rate remained higher than 20% at day 10 (FIG. 7A), but this dosage of Curcumin inhibited the cell growth slightly (FIG. 7B). Low dosage of Curcumin at luM did not enhance the transduction rate but enhanced the cell growth. The highest dosage of 100 uM could slightly enhance the transduction rate but significantly impaired the cell growth.

### Example 11. Dexamethasone Improved CAR Related Lentivirus Transduction of 762 T Cell

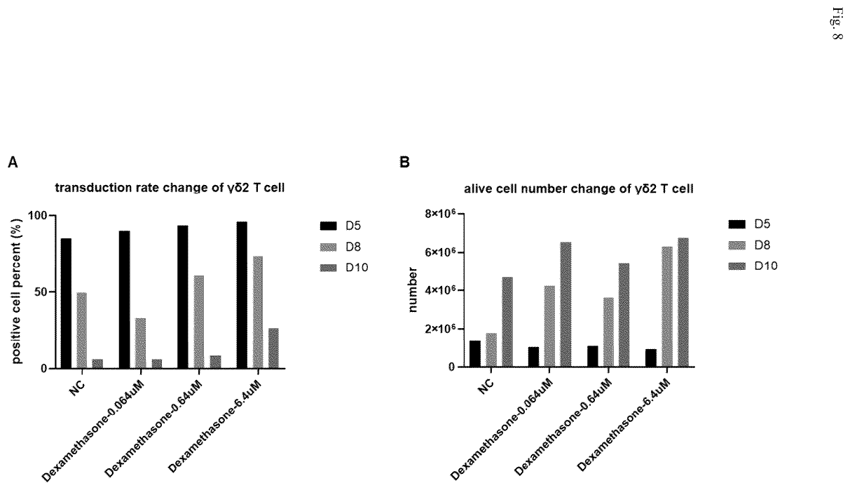

The transduction rate of the control group decreased continuously from Day5 to Day10 which was around 5%. Dexamethasone could enhance the transduction rate in a dosage dependent manner from 0.064 uM to 6.4 uM (FIG. 8A). At the dosage of 6.4 uM, the transduction rate was higher than 25%. The adding of Dexamethasone did not impair the cell growth (FIG. 8B).

### Example 12. 2-Aminopurine Improved CAR Related Lentivirus Transduction of 762 T Cell

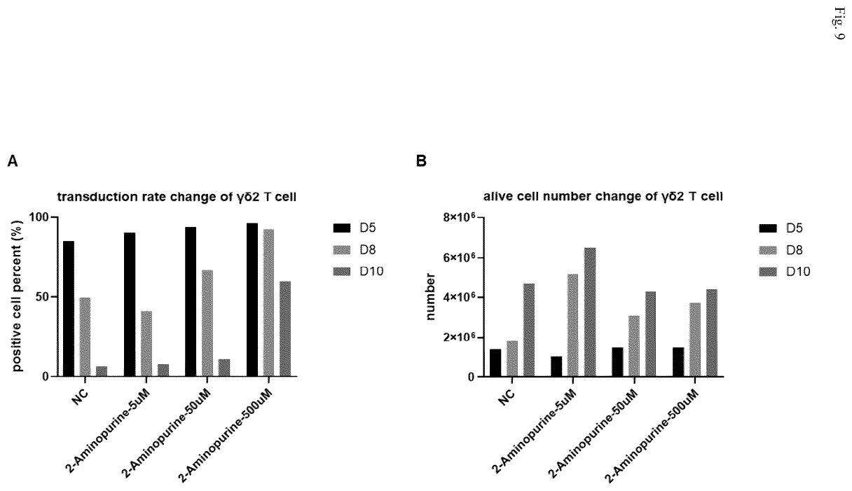

The transduction rate of the control group decreased continuously from Day5 to Day10 which was around 5%. 2-Aminopurine could enhance the transduction rate in a dosage dependent manner from 5 uM to 500 uM (FIG. 9A). At the dosage of 500 uM, the transduction rate was around 60%. The adding of 2-Aminopurine did not impair the cell growth (FIG. 9B).

### Example 13. (5Z)-7-Oxozeaenol Improved CAR Related Lentivirus Transduction of 762 T Cell

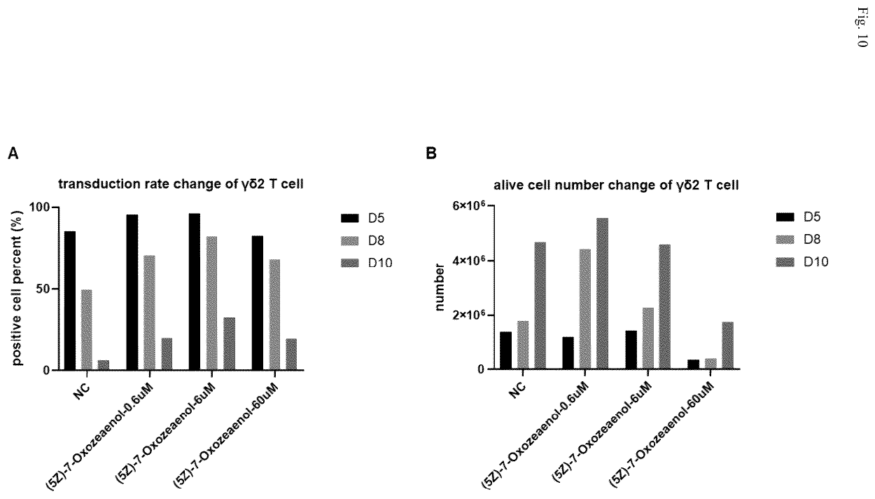

The transduction rate of the control group decreased continuously from Day5 to Day10 which was around 5%. The transduction rate with (5Z)-7-Oxozeaenol at 0.6 uM was higher than 20% and higher than 30% as the dosage reached to 6 uM (FIG. 10A). Higher dosage at 60 uM did not perform better to improve the transduction rate but impaired the cell growth than the dosage at 6 uM (FIG. 10B). The application of (5Z)-7-Oxozeaenol at the dosage of 0.6 uM and 6 uM did not influence the cell growth.

### Example 14. IRAK1/4 Inhibitor I Improved CAR Related Lentivirus Transduction of 762 T Cell

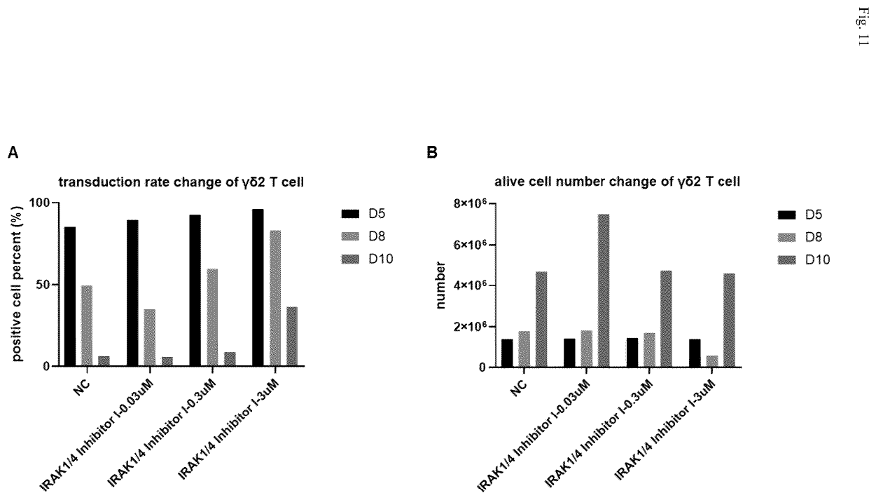

The transduction rate of the control group decreased continuously from Day5 to Day10 which was around 5%. IRAK1/4 Inhibitor I could enhance the transduction rate in a dosage dependent manner from 0.03 uM to 3 uM (FIG. 11A). At the dosage of 3 uM, the transduction rate was higher than 35%. The adding of IRAK1/4 Inhibitor I did not impair the cell growth (FIG. 11B).

### Example 15. Bortezomib Improved CAR Related Lentivirus Transduction of 762 T Cell

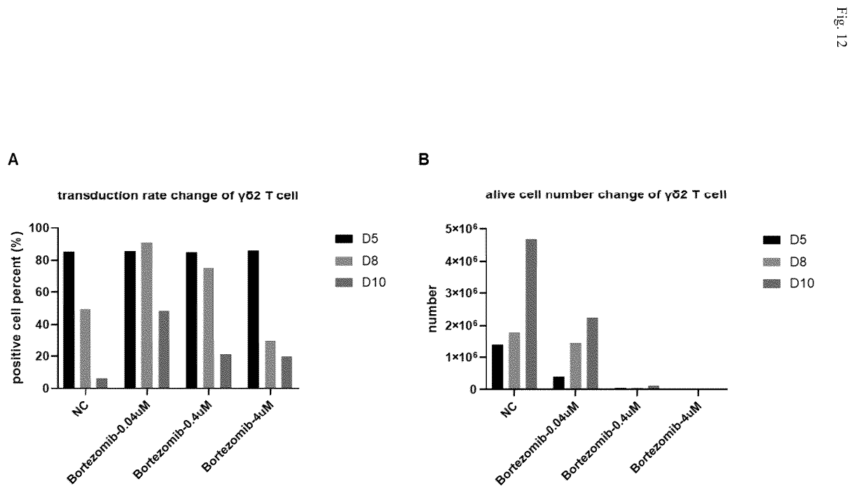

The transduction rate of the control group decreased continuously from Day5 to Day10 which was around 5%. Bortezomib could enhance the transduction rate which was around at 50% at the dosage of 0.04 uM (FIG. 12A), higher dosage (0.4 uM and 4 uM) of Bortezomib could also improve the transduction rate which was higher than 20%. The adding of Bortezomib impaired the cell growth in a dosage dependent manner and the dosage at 0.4 uM and 4 uM resulted in significantly cell number loss (FIG. 12B).

### Example 16. Small Inhibitors Improved CAR Related Lentivirus Transduction of 761 T Cell

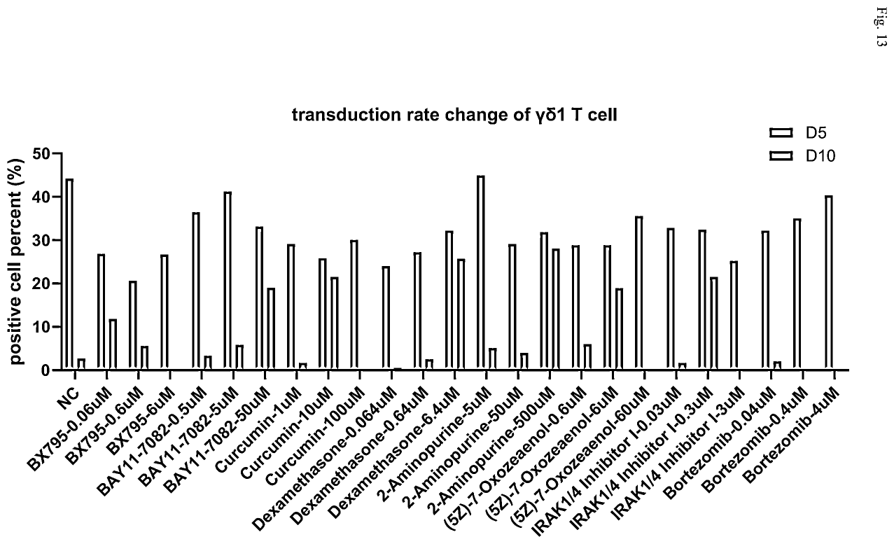

To test the application of these small molecule on improving the transduction rate of δδ1 T cells. We evaluated the transduction rate of δδ1 T cells with/without different dosage of small molecules. As can be seen from FIG. 13, the transduction rate of control group decreased dramatically from Day5 to Day10 which was finally around 2%. All the molecules except for Bortezomib could improve the transduction rate under certain concentration such as BX795-0.06 uM, BAY11-7082-50 uM, Curcumin-10 uM, Dexamethasone-6.4 uM, 2-Aminopurine-500 uM, (5Z)-7-Oxozeaenol-6 uM and IRAK1/4 Inhibitor I-0.3 uM.

### Example 17. Construction of CAR δδ2 T Targeted to CD4 and their Tumor Cell Killing Efficiency In Vitro

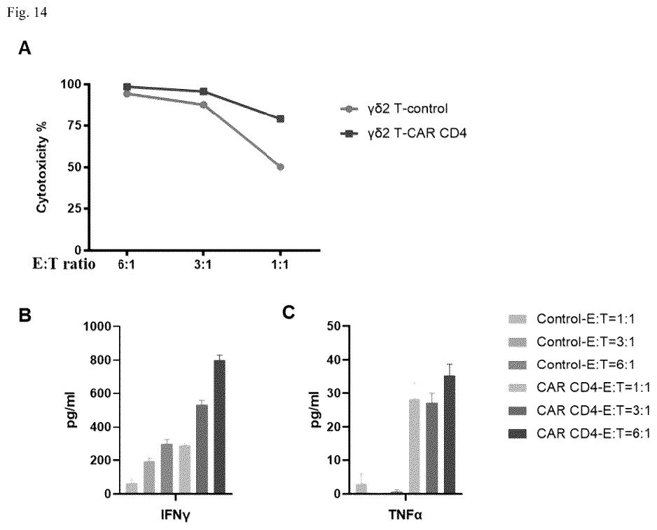

CAR γδ2 T which targeted to CD4 were constructed and their tumor cell killing efficiency were calculated in vitro. The unmodified γδ2 T cell (γδ2 T control) had a cytotoxicity to CD4 positive tumor cells (Jurkat T-luc, a human T cell leukemia cell, and the cells were stably expressed fire-fly-luciferase) in a E:T ratio dependent manner, and CAR γδ T cell (γδ2 T-CAR CD4) performed better (FIG. 14A). Two killing cytokines were monitored after the cytotoxicity test. CAR γδ2 T cell secreted much more IFNγ and TNFα than unmodified γδ2 T cells (FIGS. 14B and 14C).

### Example 18. CAR δδ2 T Targeted to CD4 Inhibited Tumor Growth In Vivo

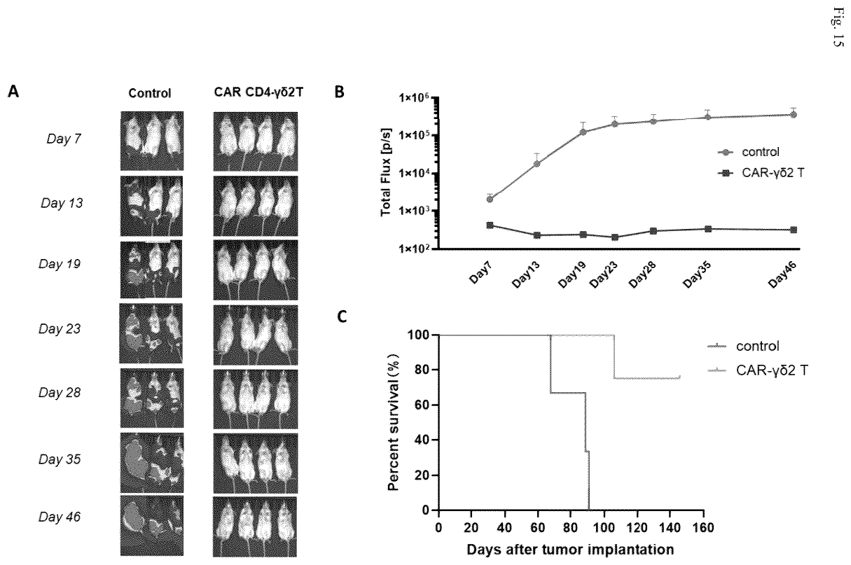

We used Jurkat T to study the tumor inhibition of CAR-CD4 γδ2 T in vivo. Jurkat T-luc tumor cells were implanted into the immune deficient mice by intravenous injection (i.v.) and 1.0×10\hat{ }6 tumor cells were given to each mice at day 0. At day 2, day 5, day 8, day 11 and day 14, 2×10\hat{ }6 CAR positive CAR-γδ2 T (CAR-CD4) were given respectively. It can be seen that CAR-γδ2 T therapy could significantly impair the tumor growth (FIG. 15A and B) and prolonged the life time of tumor bared mice (FIG. 15C).

### Example 19. CAR δδ2 T Targeted to B7H3 Inhibited Tumor Growth In Vivo

SKOV3, a human ovarian cancer was used to test the tumor inhibition ability of CAR γδ2 T cell in vivo.

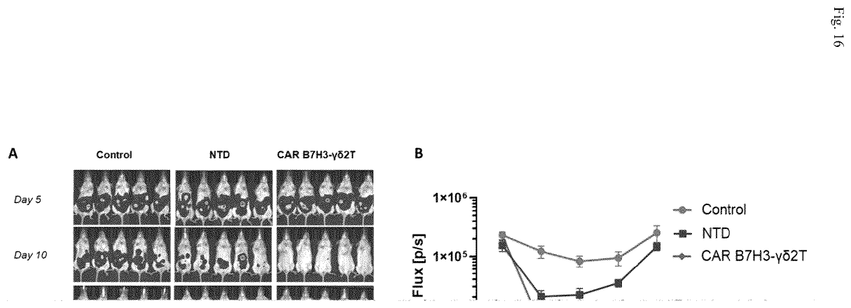

SKOV3-luc tumor cells were implanted into the immune deficient mice by intraperitoneal injection (i.p.), the SKOV3-luc cell was stably expressed fire-fly-luciferase and 1.5×10\hat{ }6 tumor cells were given to each mice at day 0. γδ2 T (NTD) or CAR-γδ2 T (CAR-B7H3) cells were given (i.p.) at day 6, day 9 and day 12 respectively, and 2×10\hat{ }6 γδ T cells were injected each time. As shown in FIG. 16, γδ2 T therapy could inhibit the growth of SKOV3 tumor and CAR-γδ2 T performed better.

It is much well studied that TANK-binding kinase 1 (TBK1) and IκB kinase ε (IKKε) regulate the activation of IRF3 and the production of type 1 interferons (IFNs), which trigger antiviral responses during viral infections(7). The compound BX795 was found to be a potent and selective inhibitor of PDK1, with an IC₅₀ of 6 nM, that block the phosphorylation of S6K1, Akt, PKCδ, and GSK3β. It has also been reported as a potent and relatively specific inhibitor of the TBK1 and IKKε complex, with an IC₅₀ of 6 and 41 nM, respectively. BX795 has been found to block the herpes simplex virus-1 (HVS-1) infection efficiently (8,9). Moreover, TBK1 and IKKε were also found to mediate the NF-κB response which regulates the release of different cytokines (10).

NF-κB pathway plays a key role in regulating the anti-virus immune responses. The activation of NF-κB signaling is mediated by a variety of signals. The inactivated NF-κB is located in the cytosol coupled with IκBa which inhibited the activation of NF-κB. Under the stimulation signal, the enzyme IκB kinase (IKK) would be activated which in turn, phosphorylates the IκBa protein, which results in the ubiquitination and dissociation of IκBa from NF-κB and results in the activation of NF-κB.

BAY 11-7082 (Catalog No.S2913, Synonyms: BAY 11-7821) is a NF-κB inhibitor, inhibits TNFα-induced IκBa phosphorylation (11). BAY 11-7082 also inhibits ubiquitin-specific protease USP7 and USP21 with IC50 of 0.19 μM and 0.96 μM, respectively. BAY 11-7082 induces apoptosis and S phase arrest in gastric cancer cells. Curcumin (diferuloylmethane) is a bright yellow chemical produced by plants of the Curcuma longa species. It has been shown to block many reactions in which NF-κB plays a major role, exhibited both anti-inflammatory, anti-bacterial/fungal/viral, anti-cancer, and anti-oxidant activities properties. Moreover, Curcumin was found to impair the NF-κB signaling by inhibiting the activation of IKK which blocked the phosphorylation of the IκBa protein (12,13).

Akt (PKB/Akt) or protein kinase B is a serine/threonine kinase, which in mammals comprises three highly homologous members known as PKBα (Akt1), PKBβ (Akt2), and PKBγ (Akt3). Akt is activated by lipid products of phosphatidylinositol 3-kinase (PI3K). Akt phosphorylates and regulates the function of many cellular proteins involved in processes that include innate/adaptive immune response, metabolism, apoptosis, and proliferation. Akt can induce the phosphorylation and lead to the degradation of IκB to regulate the activation of NF-κB (14). Dexamethasone is a glucocorticoid medication which was applied to treat different kinds of immune-disorder disease such as rheumatic problems, severe allergies, asthma and croup, et al. It has been well defined the molecular mechanism of Dexamethasone was induced reductions in Akt activity which then inhibited the NF-κB signaling (15-17).

In many cases, under immune stimulation, JNK and p38 signaling work together with NF-κB to modulate the immune response, all these three pathways are regulated by MAPK (mitogen-activated protein kinase) cascade (18,19). JNKs (c-Jun N-terminal kinases) were kinds of kinases bind and phosphorate cJun on Ser, they are belonging to the MAPK family and response to different stress stimuli to regulate the inflammatory activation. They also participate in the regulation of T cell differentiation and the cellular apoptosis pathway. p38 mitogen-activated protein kinase are also MAPK family members and respond to stress stimuli such as cytokines and UV exposure, they are also involved in cell differentiation, apoptosis and autophagy.

Protein kinase R (PKR) is a serine-threonine kinase which plays a major role in central cellular processes such as mRNA translation, transcriptional control, regulation of apoptosis, and proliferation. The dysregulation of PKR was found in cancer, neurodegeneration, metabolism and inflammatory disorders. It acts as an activator on the signaling cascades involved during stress-activated protein kinases (MAPK) action. It is located upstream of the activation of JNK, p38 and NF-κB in response to several cytokines, such as IL-I and TNF-α, and many other components (20). 2-Aminopurine, a purine analog of guanine and adenine, is used as a PKR inhibitor (21). TAK1, also known as mitogen-activated protein kinase kinase kinase 7 (MAP3K7) is an evolutionarily conserved kinase in the MAP3K family and clusters with the tyrosine-like and sterile kinase families. TAK1 can be induced by TGFbeta and morphogenetic protein (BMP), which mediates the functions in transcription regulation and apoptosis. TAK1 has been proved to mediate the cell death under both intra and extracellular stimuli. TAK1 activated by these multiple mechanisms upregulates NF-κB and AP-1-depenedent gene expression through activating the NF-κB and MAP kinase (JNK and p38) pathways (22). (5Z)-7-Oxozeaenol is a resorcyclic lactone of fungal origin that acts as a potent and selective TAK1 inhibitor (23). IRAK-1 (Interleukin-1 receptor-associated kinase 1) is an kinase enzyme belongs to IRAK family consisting of IRAK-1, IRAK-2, IRAK-3, and IRAK-4, and is activated by inflammatory molecules. IRAK1 mediates the activation of the IKK complex by cooperating with an E3 ubiquitin ligase, TRAF6, which mediates the activation of the IKK complex, resulting in the activation of NF-κB signaling. On the other hand, the IRAK1/TRAF6 complex can also activate JNK and p38 signalling through assembly of a catalytically active TAB2-TAB3-TAK1 complex (24).

Besides all the small inhibitors mentioned above, Bortezomib is another one which could inhibit the NF-κB signaling (25). Bortezomib is a targeted therapy and is classified as a proteasome inhibitor. It is an anti-cancer medication used to treat multiple myeloma and mantle cell lymphoma.

Therefore, we tested if blocking NF-κB pathway related innate immunity and anti-virus activity by different kinds of small inhibitors could prevent the induction of interferons, reduce host response, and stabilize viral transduction in γδ T cells. The small inhibitors here could be divided into several groups: 1. directly inhibit the phosphorylation of IκBa including BAY11-7082; 2. inhibit the function of IkB kinase such as Curcumin; 3. inhibit the function of TBK1 which is the upstream kinase of NF-κB pathway such as BX795; 4. inhibit the function of AKT which is the upstream kinase of NF-κB pathway such as Dexamethasone; 5. inhibit the function of NF-κB as well as p38 and JNK signaling including 2-Aminopurine, (5Z)-7-Oxozeaenol and IRAK1/4 Inhibitor I which regulate the kinases of PKR, TAK1 and IRAK1 respectively; 6. the ones that impair NF-κB activation with not known mechanism such as Bortezomib.

These experiments results demonstrated that the use of these inhibitors increased the transduction rate and also maintained the high transduction rate during subsequent cell culture and expansion.

The present disclosure is not to be limited in scope by the specific embodiments described herein. Indeed, various modifications of the subject matter provided herein, in addition to those described, will become apparent to those skilled in the art from the foregoing description. Such modifications are intended to fall within the scope of the appended claims. Various publications, patents and patent applications are cited herein, the disclosures of which are incorporated by reference in their entireties.

### REFERENCES

1. Deniger, D. C., Moyes, J. S., and Cooper, L. J. N. (2014) Clinical Applications of Gamma Delta T Cells with Multivalent Immunity. Frontiers in Immunology 5
2. M, B., K, W., and B, M. (2005) Professional antigen-presentation function by human gammadelta T Cells. Science. 2005 Jul. 8; 309(5732):264-8. doi: 10.1126/science.1110267. Epub 2005 Jun. 2.,-264-268
3. Xu, Y., Xiang, Z., Alnaggar, M., Kouakanou, L., Li, J., He, J., Yang, J., Hu, Y., Chen, Y., Lin, L., Hao, J., Li, J., Chen, J., Li, M., Wu, Q., Peters, C., Zhou, Q., Li, J., Liang, Y., Wang, X., Han, B., Ma, M., Kabelitz, D., Xu, K., Tu, W., Wu, Y., and Yin, Z. (2020) Allogeneic Vγ9Vδ2 T-cell immunotherapy exhibits promising clinical safety and prolongs the survival of patients with late-stage lung or liver cancer. Cellular & Molecular Immunology
4. Wang, R. N., Wen, Q., He, W. T., Yang, J. H., Zhou, C. Y., Xiong, W. J., and Ma, L. (2019) Optimized protocols for gammadelta T cell expansion and lentiviral transduction. Mol Med Rep 19, 1471-1480
5. Ang, W. X., Ng, Y. Y., Xiao, L., Chen, C., Li, Z., Chi, Z., Tay, J. C.-K., Tan, W. K., Zeng, J., Toh, H. C., and Wang, S. (2020) Electroporation of NKG2D RNA CAR Improves Vγ9Vδ2 T Cell Responses against Human Solid Tumor Xenografts. Molecular Therapy-Oncolytics 17, 421-430
6. Rozenbaum, M., Meir, A., Aharony, Y., Itzhaki, O., Schachter, J., Bank, I., Jacoby, E., and Besser, M. J. (2020) Gamma-Delta CAR-T Cells Show CAR-Directed and Independent Activity Against Leukemia. Frontiers in Immunology 11
7. Clark, K., Plater, L., Peggie, M., and Cohen, P. (2009) Use of the pharmacological inhibitor BX795 to study the regulation and physiological roles of TBK1 and IkappaB kinase epsilon: a distinct upstream kinase mediates Ser-172 phosphorylation and activation. The Journal of biological chemistry 284, 14136-14146
8. Jaishankar, D., Yakoub, A. M., Yadavalli, T., Agelidis, A., Thakkar, N., Hadigal, S., Ames, J., and Shukla, D. (2018) An off-target effect of BX795 blocks herpes simplex virus type 1 infection of the eye. Science translational medicine 10, eaan5861
9. Iqbal, A., Suryawanshi, R., Yadavalli, T., Volety, I., and Shukla, D. (2020) BX795 demonstrates potent antiviral benefits against herpes simplex Virus-1 infection of human cell lines. Antiviral Research 180, 104814
10. Balka, K. R., Louis, C., Saunders, T. L., Smith, A. M., Calleja, D. J., D'Silva, D. B., Moghaddas, F., Tailler, M., Lawlor, K. E., Zhan, Y., Burns, C. J., Wicks, I. P., Miner, J. J., Kile, B. T., Masters, S. L., and De Nardo, D. (2020) TBK1 and IKKε Act Redundantly to Mediate STING-Induced NF-κB Responses in Myeloid Cells. Cell reports 31
11. Pierce, J. W., Schoenleber, R., Jesmok, G., Best, J., Moore, S. A., Collins, T., and Gerritsen, M. E. (1997) Novel Inhibitors of Cytokine-induced IκBa Phosphorylation and Endothelial Cell Adhesion Molecule Expression Show Anti-inflammatory Effects in Vivo. Journal of Biological Chemistry 272, 21096-21103
12. Olivera, A., Moore, T. W., Hu, F., Brown, A. P., Sun, A., Liotta, D. C., Snyder, J. P., Yoon, Y., Shim, H., Marcus, A. I., Miller, A. H., and Pace, T. W. W. (2012) Inhibition of the NF-κB signaling pathway by the curcumin analog, 3,5-Bis(2-pyridinylmethylidene)-4-piperidone (EF31): anti-inflammatory and anti-cancer properties. Int Immunopharmacol 12, 368-377
13. Shishodia, S., Potdar P Fau-Gairola, C. G., Gairola Cg Fau-Aggarwal, B. B., and Aggarwal, B. B.

Curcumin (diferuloylmethane) down-regulates cigarette smoke-induced NF-kappaB activation through inhibition of IkappaBalpha kinase in human lung epithelial cells: correlation with suppression of COX-2, MMP-9 and cyclin D1.
14. Bai, D., Ueno, L., and Vogt, P. K. (2009) Akt-mediated regulation of NFkappaB and the essentialness of NFkappaB for the oncogenicity of PI3K and Akt. International journal of cancer 125, 2863-2870
15. Zhao, W., Qin, W., Pan, J., Wu, Y., Bauman, W. A., and Cardozo, C. (2009) Dependence of dexamethasone-induced Akt/FOXO1 signaling, upregulation of MAFbx, and protein catabolism upon the glucocorticoid receptor. Biochemical and Biophysical Research Communications 378, 668-672
16. Kim, J., Park, M. Y., Kim, H. K., Park, Y., and Whang, K.-Y. (2016) Cortisone and dexamethasone inhibit myogenesis by modulating the AKT/mTOR signaling pathway in C2C12. Bioscience, Biotechnology, and Biochemistry 80, 2093-2099
17. Ribeiro, S. B., de Araijo, A. A., Araijo Jdnior, R. F d., Brito, G. A. d. C., Leit§ o, R. C., Barbosa, M. M., Garcia, V. B., Medeiros, A. C., and Medeiros, C. A. C. X. d. (2017) Protective effect of dexamethasone on 5-FU-induced oral mucositis in hamsters. PLOS ONE 12, e0186511
18. Huang, G., Shi, L. Z., and Chi, H. (2009) Regulation of JNK and p38 MAPK in the immune system: signal integration, propagation and termination. Cytokine 48, 161-169
19. Jeong, Y. E., and Lee, M.-Y. (2018) Anti-Inflammatory Activity of Populus deltoides Leaf Extract via Modulating NF-κB and p38/JNK Pathways. International Journal of Molecular Sciences 19
20. Garcia, M. A., Gil, J., Ventoso, I., Guerra, S., Domingo, E., Rivas, C., and Esteban, M. (2006) Impact of protein kinase PKR in cell biology: from antiviral to antiproliferative action. Microbiol Mol Biol Rev 70, 1032-1060
21. Velloso, L. A. (2014) Turning Off a Viral/Lipid Sensor Improves Type 2 Diabetes. Diabetes 63, 393-395
22. Hirata, Y., Takahashi, M., Morishita, T., Noguchi, T., and Matsuzawa, A. Post-Translational Modifications of the TAK1-TAB Complex. LID-10.3390/ijms18010205 [doi] LID-205.
23. Ninomiya-Tsuji, J., Kajino T Fau-Ono, K., Ono K Fau-Ohtomo, T., Ohtomo T Fau-Matsumoto, M., Matsumoto M Fau-Shiina, M., Shiina M Fau-Mihara, M., Mihara M Fau-Tsuchiya, M., Tsuchiya M Fau-Matsumoto, K., and Matsumoto, K. A resorcylic acid lactone, 5Z-7-oxozeaenol, prevents inflammation by inhibiting the catalytic activity of TAK1 MAPK kinase kinase.
24. Rhyasen, G. W., and Starczynowski, D. T. (2015) IRAK signalling in cancer. British Journal of Cancer 112, 232-237
25. Paramore, A., and Frantz, S. (2003) Bortezomib. Nature Reviews Drug Discovery 2, 611-612

1. A method of transducing a γδ T cell with a viral vector, comprising:
contacting the γδ T cell with
i) the viral vector; and
ii) an agent capable of inhibiting the innate anti-virus activity of the γδ T cell.

2. The method of claim 1, wherein the γδ T cell is a δ1, δ2 or δ3 T cell.

3. The method of claim 1, wherein the γδ T cell is a γ9δ2 T cell.

4. The method of claim 1, wherein the viral vector is a retroviral vector or a lentiviral vector.

5. (canceled)

6. The method of claim 1, wherein the viral vector is a VSV-G pseudotyped lentiviral vector.

7. (canceled)

8. The method of claim 1, wherein the agent is an inhibitor of IKKα, IKKβ, IKKε, IκB kinase, TBK1, PKD1, NF-κB, Akt, PKR, TAK1, IRAK1/4 or proteasome.

9. (canceled)

10. The method of claim 1, wherein the agent is selected from the group consisting of BX795, BAY11-7082, Curcumin, Dexamethasone, 2-Aminopurine, (5Z)-7-Oxozeaenol, IRAK1/4 Inhibitor I, and Bortezomib.

11.-21. (canceled)

22. The method of claim 1, further comprising culturing the transduced γδ T cell in a medium without the agent capable of inhibiting the innate anti-virus activity of the γδ T cell.

23. The method of a claim 1, wherein the viral vector comprises a nucleotide sequence encoding a chimeric antigen receptor (CAR).

24. A method of preparing CAR-γδ T cells, comprising:
1) providing δδ T cells; and
2) transducing the γδ T cells with a viral vector comprising a nucleotide sequence encoding a chimeric antigen receptor in the present of an agent capable of inhibiting the innate anti-virus activity of the γδ T cells.

25. The method of claim 24, wherein said 1) comprises culturing peripheral blood mononuclear cells (PBMCs) in a medium supplemented with IL-2 and ZOL.

26. The method of claim 24, further comprising 3) culturing the transduced γδ T cells in a medium without the agent capable of inhibiting the innate anti-virus activity of the γδ T cells.

27. The method of claim 24, wherein the γδ T cell is a δ1, δ2 or δ3 T cell.

28. The method of claim 24, wherein the γδ T cell is a 7962 T cell.

29. The method of claim 24, wherein the viral vector is a retroviral vector or a lentiviral vector.

30. (canceled)

31. (canceled)

32. The method of claim 24, wherein the agent is an inhibitor of IKKα, IKKβ, IKKε, IκB kinase, TBK1, PKD1, NF-κB, Akt, PKR, TAK1, IRAK1/4 or proteasome.

33. (canceled)

34. The method of claim 24, wherein the agent is selected from the group consisting of BX795, BAY11-7082, Curcumin, Dexamethasone, 2-Aminopurine, (5Z)-7-Oxozeaenol, IRAK1/4 Inhibitor I, and Bortezomib.

35. The method of claim 24, wherein the viral vector is a VSV-G pseudotyped lentiviral vector.

36.-46. (canceled)

47. A preparation comprising CAR-γδ T cells prepared by the method of a claim 24.

48. The preparation of claim 47, wherein the CAR-γδ T cells express a CAR comprising an antigen-binding domain targeting to CD4 or B7H3.

49.-52. (canceled)
